(19) United States
(12) Patent Application Publication
(10) Pub. No.: US 2010/0022804 A1
(43) Pub. Date: Jan. 28, 2010

(54) PROCESS FOR THE PRODUCTION OF FLUORINATED AROMATIC RINGS BY SIMULTANEOUS COOLING AND MICROWAVE HEATED HALOGEN EXCHANGE

(75) Inventor: BAMIDELE A. OMOTOWA, IDAHO FALLS, ID (US)

Correspondence Address: DYKAS, SHAVER & NIPPER, LLP, P.O. BOX 877, BOISE, ID, 83701-0877, US

(73) Assignee: Pearhill Technologies LLC

(21) Appl. No.: 12/355,255

(22) Filed: Jan. 16, 2009

Related U.S. Application Data
(60) Provisional application No. 61/011,210, filed on Jan. 16, 2008.

(51) Int. Cl. C07C 381/00

(52) U.S. Cl. 568/74

(57) ABSTRACT

Shown is an improved method of adding fluorine atoms to aromatic rings, using microwave energy application and simultaneous cooling to enhance the fluorination process. The result is an energy efficient method of microwave-assisted halogen exchange (HALEX) reactions involving chloroaromatics and fluorinating agents, with the result being the addition of fluorine atoms into aromatic rings.

### CROSS-REFERENCE TO RELATED APPLICATIONS

This application claims the priority date of the provisional application entitled Halex Microwave Fluorination: Energy Efficient Production of Fluorinated Active Pharmaceutical Ingredients by Halogen Exchange Processes filed by Bamidele Omotowa on Jan. 1, 2008 with application Ser. No. 61/011,210.

### FIELD OF THE INVENTION

The invention relates generally to a method for fluorinating organic compounds, and more particularly to a fluorination process which uses microwave energy with simultaneous cooling.

### BACKGROUND OF THE INVENTION

Several functional groups contribute to the bioactivity of pharmaceutical ingredients. However, the unique properties of fluoroaromatic organic compounds have increasingly proven useful in applications related to life sciences, particularly in pharmaceutical and crop-protection fields. In the $60 billion pharmaceutical industry, about 20 percent of all drugs manufactured today contain at least one fluorine atom, and thus the efficient manufacture of fluoroaromatic organic compounds is commercially important. These include highly profitable drugs like ARTOVASTATIN (PROZAC) (cholesterol medication), LANSOPRAZOLE (PREVACID) (ulcer and acid reflux treatment), FLUTICASONE PROPIONATE (FLONASE) (anti-asthma agent), and FLUXETINE (an antidepressant agent), FLUVOXAMINE (an antidepressant), EFFAVIRENZ (antiretroviral therapy for HIV), MEFLOQUINE (anti-malarial). Other drugs made from fluoroaromatics feedstocks include the fluoroquinolones, like CIPROFLOXACIN, MOXIFLOXACIN, and GATIFLOXACIN. Fluoro substitution typically improve the metabolic stability, acidity or basicity, lipophilicity, and enzyme inhibitors properties of new clinically valuable compounds, and are highly desired properties in new drugs.

New formulations are continuously being evaluated and it is predicted that over 33 percent of pharmaceuticals drugs would be fluorinated in the near future. There is obviously potential for growth of this sector of the pharmaceutical industry. Therefore, producers of the active pharmaceutical ingredients continue to build their capacity to produce the largest number of potential feedstock for short notice supply at the lowest price. As a result of this development, U.S. fine chemical companies are expanding by acquiring new chemistries that offer cheaper production costs and access to large scale manufacturing of new drugs. Since the proportion of fluorinated drugs has continued to increase, it is understandable that researches into new fluorination technologies are very high on the priorities of these businesses. It is believed that the process of the invention will be useful in this field. Table 1 presents the scale of global production of some fluoroaromatics by Halex processes in 2005.

TABLE 1
Global production of some fluoroaromatics by Halex processes in 2005

| | | Approx. global |
|---|---|---|
| | | capacity |
| Compound | Manufacturing process | (metric tons) |
| 4-Chlorobenzotrifluoride | Halogen exchange | 10,000-15,000 |
| | reaction | |
| Benzotrifluoride | Halogen exchange | 10,000 |
| | reaction | |
| Fluorobenzene | Halogen exchange | 5,000 |
| | reaction | |
| 2-Chloro-5- | Halogen exchange in HF | 1,000 |
| trifluoromethylpyridine | | |
| 2,4-Difluoroaniline | Halogen exchange | 400-600 |
| | reaction | |
| 2,6-Tifluoro-3,4,5- | Halogen exchange in HF | 500 |
| trichloropyridine | | |

Also, over the past 15 years, the number of fluorine-containing agrochemicals has grown from 4 percent to about 9 percent of the overall agrochemical production and sales. The trifluoromethyl (CF₃) group is perhaps the most significant fluoro functional constituent among the new agrochemicals. About 48 percent of them are employed as herbicides, 23 percent as insecticides, and 18 percent as fungicides.¹³ These include NORFLURAZON and FLURIDONE (herbicides), FLURPRIMIDOL (plant regulator), FLUOTRIMAZOLE and FLUTRIAFOL (fungicides).

Citing research performed by IVA, a German agrochemical industry association, Agrow, a major business publication of the agrochemical industry reported global sales of agrochemicals in 2005 at US $32.2 billion, were 12.6% higher than the year before. A growth of this trend was predicted to continue. The 2001-2002 sales records of the six largest agrochemicals producers are shown in Table 2.

TABLE 2
Reported recent agrochemicals sales

Sales (US$ Billion)

| Agro Company | 2001 | 2002 |
|---|---|---|
| Syngenta | 5.385 | 5.26 |
| Bayer | 3.978 | 3.775 |
| Monsanto | 3.755 | 3.088 |
| BASF | 3.105 | 2.787 |
| Dow | 2.612 | 2.717 |
| DuPont | 1.814 | 1.793 |

### SUMMARY OF THE INVENTION

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

The invention is an improved method of adding fluorine atoms into aromatic rings. The method of the invention uses microwave energy application and simultaneous cooling to enhance the fluorination process. The result is an energy efficient method of microwave-assisted halogen exchange (HALEX) reactions involving chloroaromatics and fluorinating agents, with the result being the addition of fluorine atoms into aromatic rings. The process is particularly useful for adding a pentafluorosulfanyl (SF₅) group to a benzene ring. Other fluorination reactions and products are clearly possible with the process of the invention, and are within the scope of the invention.

Points of Novelty of the Claimed Process:
1. Energy efficient preparation of fluorinated aromatics is achieved by a microwave heated halogen exchange (MAHE) fluorination process, combined with simultaneous cooling of the reagents.
2. The MAHE fluorination process of the process for production of fluoroaromatics is influenced by several factors that include microwave absorbing solvent, process time, microwave energy applied, catalyst, and batch, which are defined herein.
3. Process optimization by control of these factors in MAHE fluorination is used to achieve high yields of pentafluorosulfanyl fluoroaromatics products in a shorter period than has been reported for conventional halogen exchange (HALEX) processes that is based on heat conduction or microwave heating without simultaneous cooling.
4. Solvent choice for MAHE fluorination is a powerful tool for control of process heating rate and maximum achievable temperature. While the combination of this choice and program temperature setting is excellent for control of maximum process temperature, the MAHE fluorination is influenced greatly by application of high microwave power.
5. By applying maximum possible microwave power, in combination with simultaneous cooling of the reactor by a strong jet of pressurized air or by other active cooling means, the MAHE fluorination process can occur while the solvent has not achieved the set temperature.
6. The phase transfer catalyst is typically a thermally stable neutral 18-Crown-6, tetraalkylammonium, teteraalkylphosphonium, tetraphenylphosphonium, or ionic liquids, onium salts, delocalized cations, 2-azaallenium, carbophosphazenium, aminophosphonium and diphosphazeium salts, etc.
7. The process can be catalyzed by either a single phase transfer catalyst or a combination of two or more catalyst candidates to achieve an aggregate effect.
8. The pentafluorosulfanyl (SF₅) substituent is stable to microwave energy applications in an environment with <5% ionic catalyst content, in a process at about 200° C.
9. The MAHE fluorination process can be carefully composed to achieve <5% undesirable byproduct.
10. MAHE fluorination can be used to effectively achieve energy-efficient high yield production of fluoroaromatic compounds, including benzenes, naphthalene's, etc.

In the electromagnetic spectrum, microwaves (0.3-300 GHz, i.e. wavelengths range of 90 cm and 1 mm) lie between the radiowave frequency (RF) and infrared (IR) frequency and have relatively large wavelengths. In the everyday application of its properties, a microwave oven (see FIGS. 1 and 2) works by passing microwave radiation, usually at a frequency of 2.45 GHz, a wavelength of 12.24 cm, through the food. Water, fat, and sugar molecules in the food absorb energy from the microwave beam in a process called dielectric heating. Many molecules have electric dipoles, meaning that they have a positive charge at one end and a negative charge at the other, and therefore rotate as they try to align themselves with the alternating electric field induced by the microwave beam. This molecular movement creates heat as the rotating molecules hit other molecules and put them into motion.

Microwave radiation is non-ionizing and therefore, incapable of breaking bonds. Microwaves are manifested as heat through their interaction with a medium or material. They can be reflected (by non polar metals, and compounds with no dipole moment, such as CCl₄, SbF₃ and AlF₃), transmitted (by good insulators, which will not heat, such as glass), or absorbed (by organic materials) resulting in decreased available microwave energy. Absorption of microwave energy results in rapid heating of the material.

Direct microwave heating can reduce chemical reaction times from hours to minutes, and it is also known to reduce side reactions, increase yields and improve reproducibility. Therefore, academic and industrial research groups are using microwave assisted organic synthesis as a forefront technology for rapid reaction optimization, for the efficient synthesis of new chemical entities, or for discovering and probing new chemical reactivity.

Microwave heating can have effects that are different from conventional heating techniques. There is focus on what in the reaction mixture is actually absorbing the microwave energy. Materials or components of a reaction mixture can differ in their ability to absorb microwaves. Reaction rates can be increased by increasing the temperature of the reactants, delivering microwave energy faster than the heat can be transferred to the bulk solvent and radiated to the environment. For this effect to be sustainable, careful attention must be paid to vessel design and vessel cooling. This effect can be achieved using microwave reflux techniques.

Microwave irradiation does not affect the activation energy, but provides the momentum to overcome this barrier and complete the reaction more quickly than conventional heating methods. Microwave energy is related to the temperature parameter in the Arrhenius equation that describes kinetic reaction rates for chemical reactions.

K=Ae−^{Ea/RT}

Where K=Rate constant; Ea=Activation energy; R=Gas constant; T=Reaction temperature

An increase in temperature causes molecules to move about more rapidly, which leads to a greater number of more energetic collisions. This occurs much faster with microwave energy, due to the high instantaneous heating of the substance(s) above the normal bulk temperature, and is the primary factor for the observed rate enhancements. The level of instantaneous heating will be dependent on the amount of microwave energy that is used to irradiate the reactants. The higher the level of microwave energy, the higher the instantaneous temperature will be relative to the bulk temperature. One method for increasing the microwave energy that is delivered is to use simultaneous cooling during the microwave irradiation. This allows a higher level of microwave power to be directly administered, but will prevent overheating by continuously removing latent heat. This technique has proven to be very effective in further enhancing of reaction rates and will be discussed in greater detail throughout the book.

Many chloroaromatic reagents have dipole moments, and are expected to absorb microwave radiations to heat up rapidly up to slightly above their boiling points. Earlier work on microwave-assisted Halex reactions demonstrated that the phase transfer catalysts were microwave safe up to 200° C. for brief periods. When the thermosensor for the reaction measures the temperature of the liquid reagent (in this case the chlorocarbon), it is possible to control the temperature of reaction by regulating the microwave energy required to achieve a set temperature. Some broad potential benefits of this research proposal are highlighted in the following discussions on the pharmaceutical drugs and agrochemicals production.

The process of the invention has been demonstrated to achieve energy efficient microwave-assisted halogen exchange (MAHE) reactions of haloaromatics and solid inorganic fluorinating agents for introducing fluorine atoms into aromatic rings. Fluorinated aromatics are huge synthetic ingredients for the production of fine pharmaceuticals and agrochemicals, and have annual market worth estimated at more than US $4 billion. As an example of haloaromatics in general, the conventional endothermic halogen exchange (HALEX) process currently used in industry for production of fluoroaromatic compounds that do not contain pentafluorosulfanyl (SF₅) group, expends significant amount of energy estimated at several trillion BTU/year.

Those processes involve the reaction of a solid inorganic fluoride, such as KF, CsF and RbF, with a haloaromatic reagent, and a phase transfer catalyst (PTC) at 140-260° C. As a result of low solubility of these hygroscopic fluorides in aromatic substrates, aprotic solvents, rigorous pre-reaction drying of all components, high temperature reaction conditions, and long reaction periods, as much as 9-28 h, are required to increase the concentration of fluoride in solution and reaction efficiency. This provides opportunity for many side reactions and the formation of decomposition products. By employing energy-efficient microwave process, shorter reaction periods are envisaged for the production of these fluorinated pharmaceutical ingredients, and decomposition and side reactions can be reduced significantly.

Generally, dielectric constant and dielectric loss properties of the constituent of a microwave system determine heating rate, and control of reaction conditions. The current process demonstrates the suitability and safety of traditional components of HALEX process for microwave operations (KF, haloaromatic reagents, and phase transfer catalysts).

Previously, microwave-assisted halogen exchange reactions involving KF as an agent for fluorination of basic aromatic rings was discussed in the Journal of Fluorine Chemistry (vol. 125, p. 701-704, 2004). With the application of 350 W power, the authors claimed that it was possible to achieve 90 percent yield under 3 h in small scale Halex reactions catalyzed by a polymeric phase transfer catalyst, and KF as the fluorination agent. However, the basicity of KF in the reaction medium caused the initiation of significant decomposition and formation of side products. The MAHE fluorination in this work is a careful application of this technique for novel efficient production of pentafluorosulfanyl fluoroaromatic compounds.

Still other features and advantages of the present invention will become readily apparent to those skilled in this art from the following detailed description describing preferred embodiments of the invention, simply by way of illustration of the best mode contemplated by carrying out my invention. As will be realized, the invention is capable of modification in various obvious respects all without departing from the invention. Accordingly, the drawings and description of the preferred embodiments are to be regarded as illustrative in nature, and not as restrictive in nature.

### BRIEF DESCRIPTION OF THE DRAWINGS

FIG. 1 shows the reflux condenser set-up for microwave

FIG. 2 is a diagram of microwave energy.

FIG. 3 is a diagram showing microwave heating of a liquid.

FIG. 4 is a diagram showing Conductive heating of a liquid.

FIG. 5 is a diagram showing exemplary fluorinated groups.

FIG. 6 is a diagram showing the preparation of some derivative pentafluorosulfanylbenzene compounds.

FIG. 7 is a diagram showing preparation of some derivative pentafluorosulfanylbenzene compounds.

FIG. 8 shows the preparation of pentafluorosulfanyl nitrobenzene and its derivatives.

FIG. 9 shows the preparation of pentafluorosulfanyl haloaromatics by formation of sulfur chlorotetrafluoride intermediate.

FIG. 10 shows ROCHE's patented APIs containing pentafluorosulfanyl (SF₅) group

FIG. 11 shows the process for preparation of Levofloxacin hemihydrates employs tetrafluorobenzoic acid as starting material

FIG. 12 shows the reaction vessel and microwave heater of the invention.

FIG. 13 shows the addition of reagents to the reaction vessel.

FIG. 14 shows the reflux cooler on the reaction vessel.

FIG. 15 shows the reactants being stirred with gas purging.

FIG. 16 shows a pressurizable reaction vessel.

FIG. 17 shows the production of pentafluorosulfanyl 4-halobenzene by direct fluorination of bis(4-halophenyl) disulfide.

FIG. 18 shows the MAHE fluorination of pentafluorosulfanyl 4-bromobenzene.

### DESCRIPTION OF THE PREFERRED EMBODIMENTS

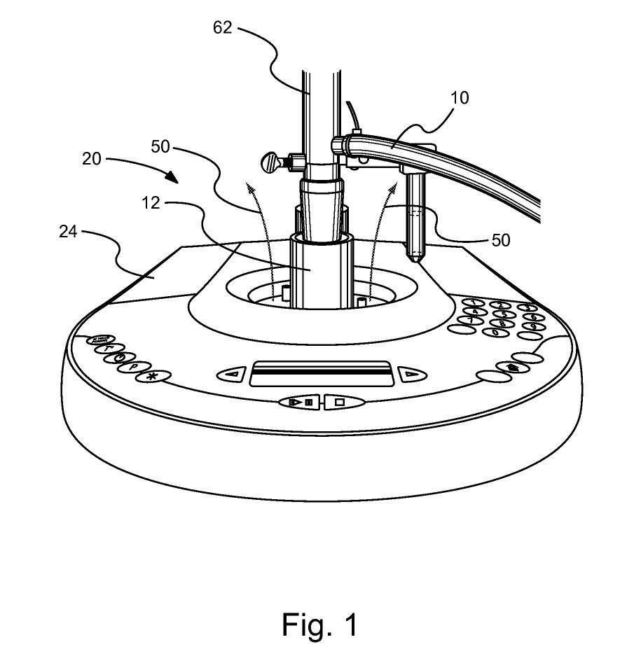

The invention is an improved method of adding fluorine atoms into aromatic rings, and is further described in FIGS. 1-18. The method of the invention uses microwave energy application and simultaneous cooling to enhance the fluorination process. The result is an energy efficient method of microwave-assisted halogen exchange (HALEX) reactions involving chloroaromatics and fluorinating agents, with the result being the addition of fluorine atoms into aromatic rings. The process is defined by the claims, which are broader in scope than the description of the preferred embodiment. Since a description of at least one preferred embodiment is required by patent rules, the following is a description of a preferred embodiment of the process, in which a pentafluorosulfanyl (SF₅) group is added to a benzene ring. Other fluorination reactions and products are clearly possible with the process of the invention, and a detailed description of the process for attaching this specific group is meant to be illustrative of the process, and not to be more limiting than the claims.

Microwave-assisted Halex reactions involving KF as fluorinating agent were discussed in a paper published in the Journal of Fluorine Chemistry in 2004. With the application of 350 W power, the authors claimed that it was possible to achieve 90 percent yield under 3 hour in small scale Halex reactions catalyzed by a polymeric phase transfer catalyst, and KF as the fluorination agent. However, the basicity of KF in the reaction medium caused the initiation of significant decomposition and formation of side products.

The conventional endothermic Halex process currently used in industry expends a significant amount of energy, estimated at several trillion BTU/year for all production. Those processes involve the reaction of a solid inorganic fluoride, such as KF, CsF and RbF, with a chloroorganic reagent, and a phase transfer catalyst (PTC) above 200° C. As a result of low solubility of these hygroscopic fluorides in chloroorganic reagents, aprotic solvents, rigorous pre-reaction drying of all components, high temperature reaction conditions (above 240° C.), and long reaction periods are required to increase the concentration of fluoride in solution and reaction efficiency. This provides opportunity for many side reactions and the formation of decomposition products. To improve on this process, the process of the invention employs acid fluorides in a microwave heating process, by which decomposition and side reactions, solvents, and PTCs are avoidable, and shorter reaction periods are envisaged and far less energy is used in the production of fluorinated pharmaceutical ingredients.

The method of the invention focuses on adding a fluorine atom to an aromatic molecule. Of particular applicability to the process is the addition of a pentafluorosulfanyl (SF₅) group to an organic molecule. The impetus for creating new SF₅-bearing new compounds is related to the fact that the SF₅ group possesses a greater electronegativity than the trifluoromethyl group (CF₃) and as a result, the SF₅ addition is thought to represent an advantageous alternative to the well-established and widely used practice of creating compounds bearing the CF₃ moiety. In a manner that is analogous to CF₃-bearing organic compounds, SF₅ has been described by an expert in the field as the “substituent of the future”. Yet to be synthesized compounds containing the “SF₅” moiety may likely represent the next wave of highly useful fluorochemicals that will be further distinguished over their predecessor CF₃-bearing compounds by their outstanding chemical properties. These outstanding properties may include: high to extreme chemical and thermal stability, oleophobicity, lipophilicity, high density, low polarizability and low surface tension. It is anticipated that new fluorine bearing organic compounds in general, and SF₅-bearing organic compounds in particular, will be utilized as new and potent pharmaceuticals, pesticides, herbicides, antibiotics, blood substitutes, fungicides, specialty polymers, lubricants, liquid crystals, and surfactants. The difficulty of commercializing such SF₅-bearing organic compounds is in part related to the difficulty of obtaining sufficient and affordable quantities of any number potentially useful SF₅-bearing intermediate and end-product compounds.

FIG. 1 is a Reflux condenser set-up for microwave processing at atmospheric pressure under inert atmosphere. Shown in FIG. 1 is the reaction assembly 10, with a microwave heater 24 surrounding a reaction vessel 12. Attached to the reaction vessel 12 is the cooling system 20 in the form of a flow of cooled air 50. A Reflux cooler 62 with in inlet 40 is attached to the reaction vessel 12. The microwave heater supplies energy to the reactants simultaneously with the cooled air removing heat from the reaction vessel.

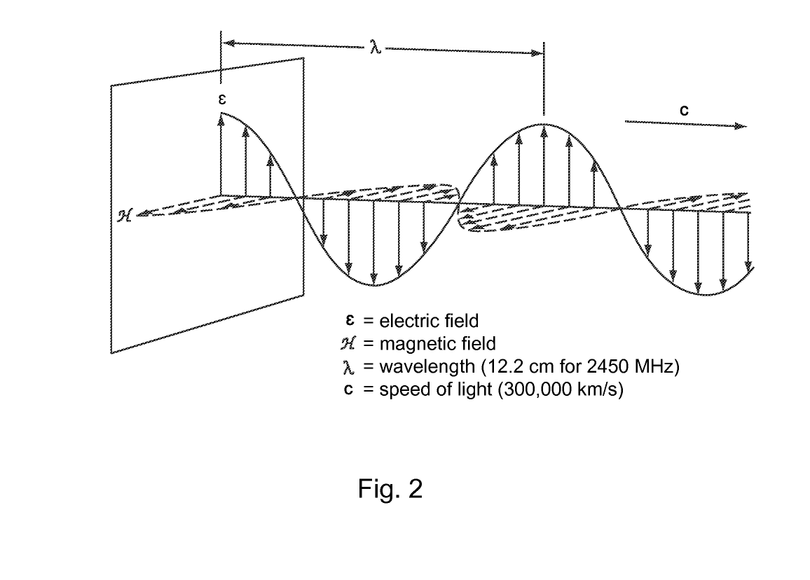

FIG. 2 is an illustration of microwave energy, showing the electric field [symbol] , the magnetic field (insert correct H shaped symbol here), the wavelength (insert correct symbol here), and the speed of light c, to illustrate the type of energy the microwave heater 24 directs at the reaction vessel 12.

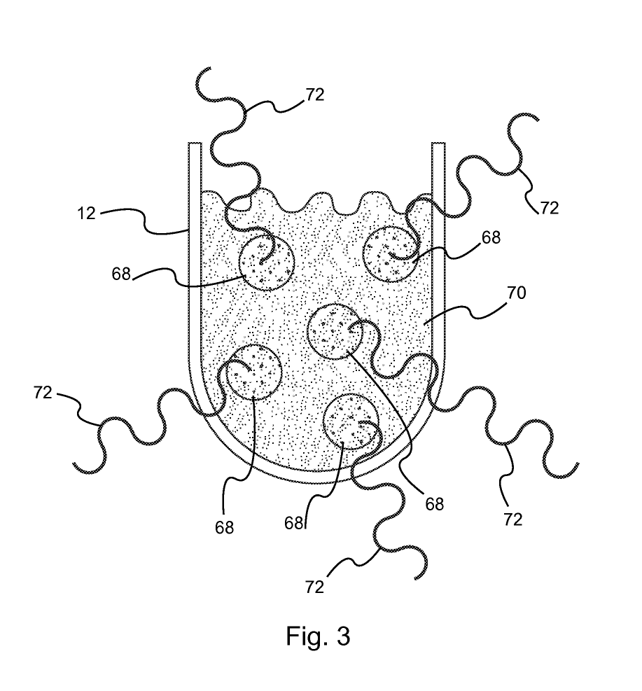

FIG. 3 shows the heating mechanism of microwave heating of solutions in which individual molecules of reactants in a reaction vessel 12 are heated by microwave energy 72. In this example the reaction vessel 12 can be glass, for instance, which is transparent to microwave energy. In such a setup, the microwave energy 72 directly activates the molecules in the mixture of reactants and solvent 70, with the result being localized superheating shown at 68. With microwave heating, which there is an instant dissipation of energy available for chemical process immediately following a switch off of the applied microwave power.

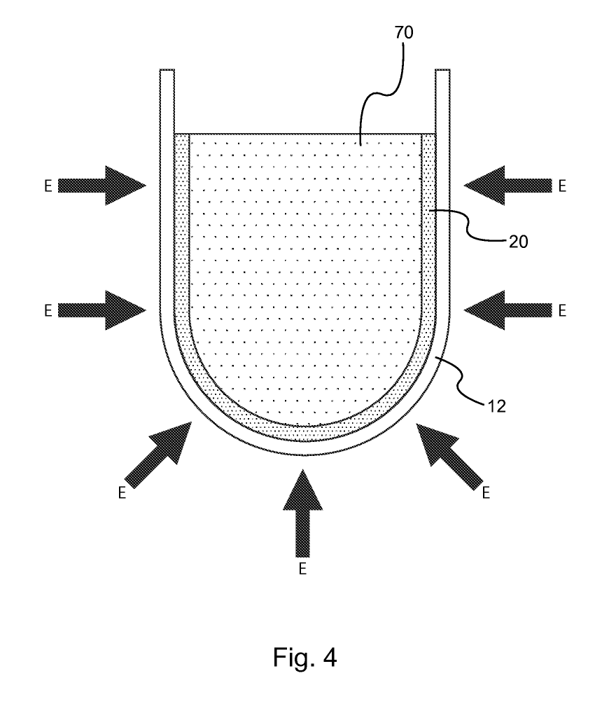

This in contrast to the conductive heating mechanism shown in FIG. 4, in which energy is transferred through the reactor vessel 12, and then dissipates through the reaction solution 70 by conduction. The reaction solution 70 nearest the vessel wall is thus hotter than solution closer to the center of the solution. After discontinuation of heat application, the reaction mixture must slowly cool to room temperature, and different mechanisms can lead to byproduct formation during this stage.

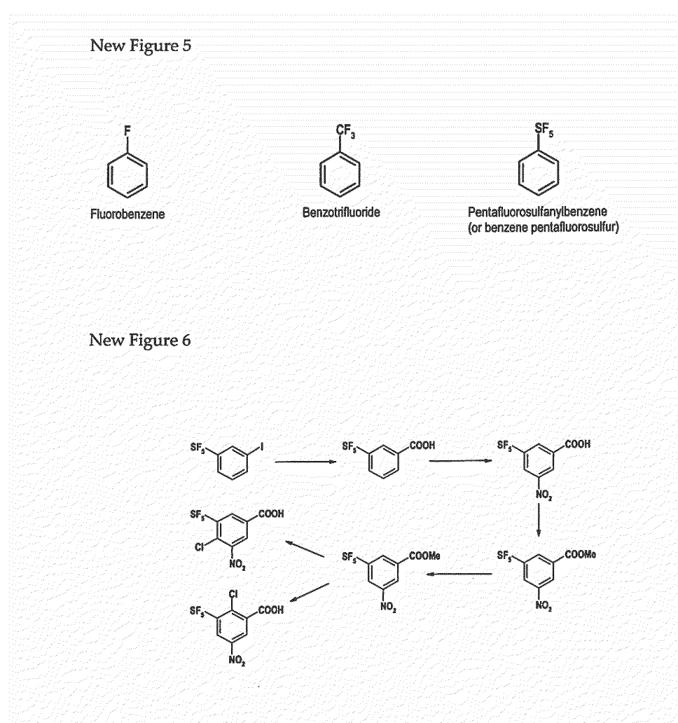

Several functional groups contribute to the bioactivity of pharmaceutical ingredients. However, the unique properties of fluoroaromatic organic compounds have increasingly endeared them to application in life sciences, particularly in pharmaceutical and crop-protection fields. To the extent that about twenty percent of all drugs manufactured today contain at least one fluorine atom in the $60 billion pharmaceutical industry. Fluoro substitution (FIG. 5) typically improves the metabolic stability, acidity or basicity, lipophilicity, and enzyme Inhibitors properties of new clinically valuable compounds, and are highly desired properties in new drugs.

FIG. 5 shows several examples of Fluoro substitution, which typically improves the metabolic stability, acidity or basicity, lipophilicity, and enzyme Inhibitors properties of new clinically valuable compounds, and are highly desired properties in new drugs. Different fluorinated groups have different effects on the electronegativity o-, m-, and p-aryl carbon atoms, lipophilicity, and pharmacological properties of pharmaceuticals.

### Examples of Fluorinated Groups.

The process of the invention has applicability to attachment of all fluorine groups to organic molecules, but attachment of the pentafluorosulfanyl (SF₅) group is a particular focus of the process of the invention. The pentafluorosulfanyl is more sterically demanding, more lipophilic, and more electronegative than the trifluoromethyl (CF₃) group on many current fluorinated drugs, the SF₅ group is virtually stable on any kind of benzene ring. Since the first synthesis of the first organic derivatives in 1960, the problem was in putting SF₅ groups onto organic structures. The rationale for creating new SF₅-bearing compounds is related to the fact that the SF₅ group possesses a greater electronegativity than the CF₃ group, and as a result, the SF₅ substitution is thought to be an advantageous alternative to the well-recognized and widely used CF₃ substituted bioactive compounds. In a manner that is analogous to CF₃-bearing organic compounds, SF₅ has been described by experts in the field as the substituent of the future. New compounds containing the SF₅ moiety represent the next wave of highly useful fluorochemicals that will be further distinguished by their superior chemical properties over the CF₃-bearing predecessor compounds first known in the 1920s. The pentafluorosulfanyl fluoroaromatics are excellent starting materials for custom synthesis of novel SF₅-compounds with outstanding properties like high to extreme chemical and thermal stability, lipophilicity, unique combination of high-density and low boiling point. They are likely to find application as pharmaceuticals, agrochemicals, advanced materials, lubricants, liquid crystals, surfactants, and specialty polymers.

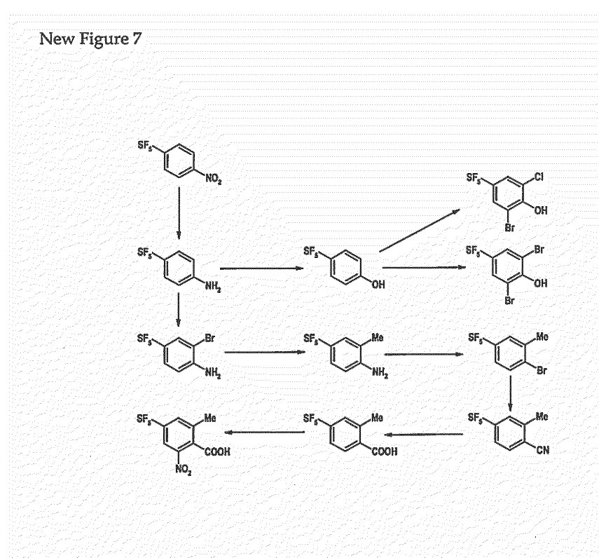

In 2008, researchers at Sanofi-Aventis Deutschland GmbH published Ortho-substituted pentafluorosulfanylbenzenes, process for their preparation and their use as valuable synthetic intermediates in U.S. Pat. No. 7,317,124 B2 (authors: Kleeman, H. W., and Week, R). Some of these reactions are illustrated in FIGS. 6 and 7.

FIG. 6 shows several prior art processes for forming pentafluorosulfanylbenzene compounds.

FIG. 7 shows several prior art processes for forming pentafluorosulfanylbenzene compounds.

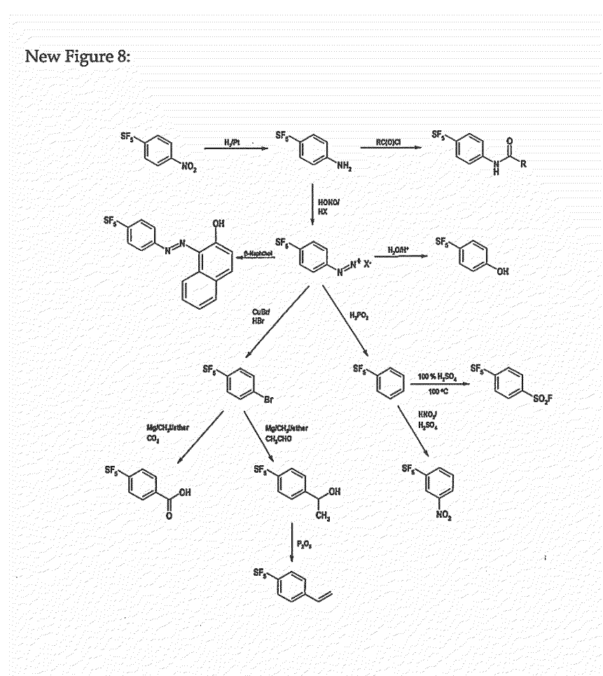

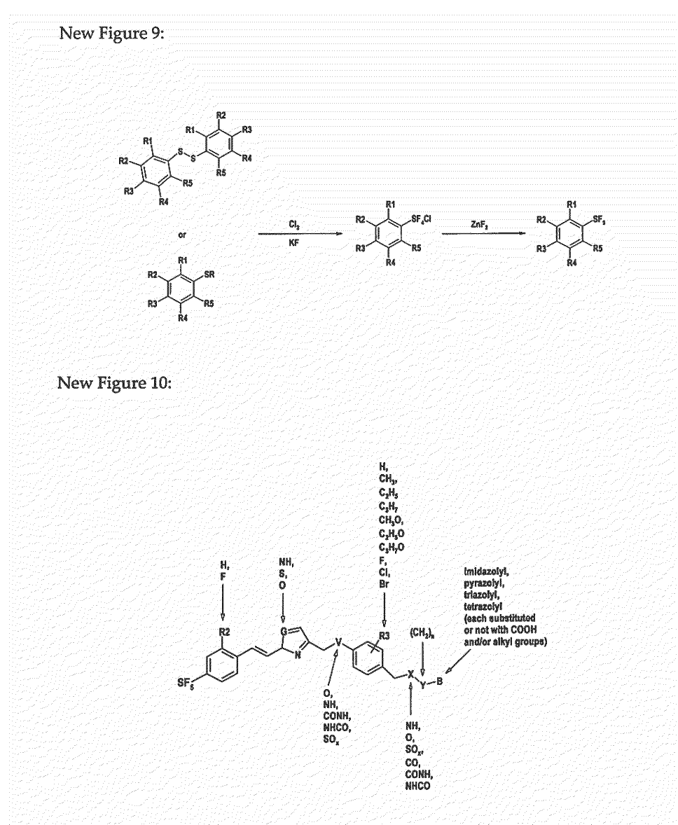

Some other derivative compounds were prepared in the published work of Dr. W. A. Sheppard (1960) in FIG. 8, F2Chemicals (2000), and IM&T Research Inc (2008) in FIG. 9. Also, ROCHE pharmaceuticals in Switzerland published APIs, shown in FIG. 10, that contain the pentafluorosulfanylbenzene moieties in 2005. These reports highlight the growing significance of this class of compounds, and there is opportunity to patent and manufacture them by novel cost effective production processes—a main objective of this proposal.

FIG. 8 shows several prior art processes for forming pentafluorosulfanyl nitrobenzene.

The first report of preparation of pentafluorosulfanyl nitrobenzene and its derivatives was in 1960.

FIG. 9 shows a prior art process for pentafluorosulfanyl haloaromatic by formation of sulfur chlorotetrafluoride intermediate.

FIG. 10 shows a prior art (ROCHE) process containing pentafluorosulfanyl groups.

By combining microwave processes and halogen exchange, the process of the invention enables large scale production of specialty fluoroaromatic compounds with the same cost and efficiency benefits that large pharmaceutical players are currently experiencing with the production of other compounds via microwave chemistry. This microwave assisted halogen exchange (MAHE) fluorination technology will not only help to reduce energy consumption by up to 50% verse conventional heating processes, but will reduce the overall carbon footprint and overall production cost. Additionally the process of the invention allows current conventional production reactors to be retrofitted with the microwave process, thereby reducing costs associated with retooling or new facility development.

Pentafluorosulfanyl aromatic compounds can be produced by as many as five different methods, but other processes are not able to prepare pentafluorosulfanyl chloroaromatic compounds in a single step. The process of the invention achieves single step production of pentafluorosulfanyl chloroaromatic compounds. Adding elemental fluorine directly to bis(chloroaromatic) disulfide or bis(polychloroaromatic) disulfide at subzero temperatures produced the respective pentafluorosulfanyl chloroaromatic or polychloroaromatic compound in high yield, in a single step.

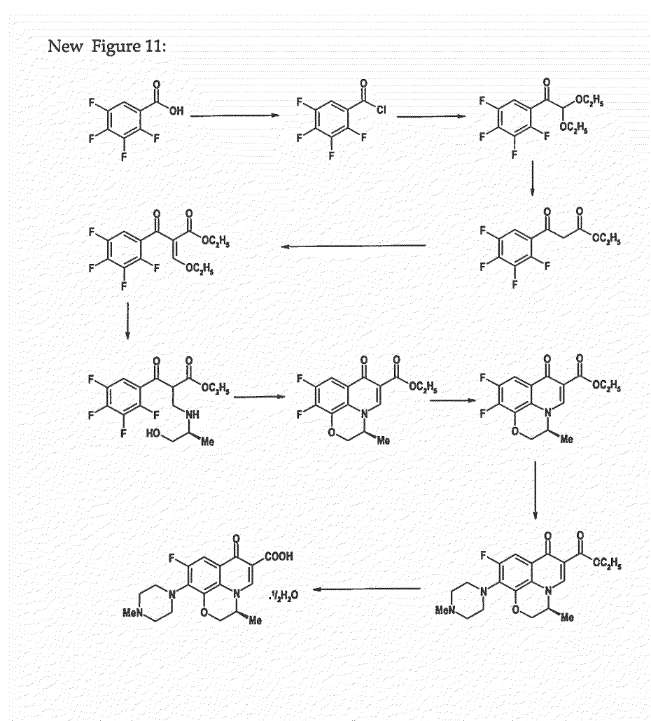

One of many potential products of the current process is exemplified by the preparation of active pharmaceutical ingredients, e.g. levofloxacin, containing SF₅ group is illustrated in FIG. 11.

FIG. 11 shows a prior art process for preparation of Levofloxacin hemihydrates employing tetrafluorobenzoic acid as starting material

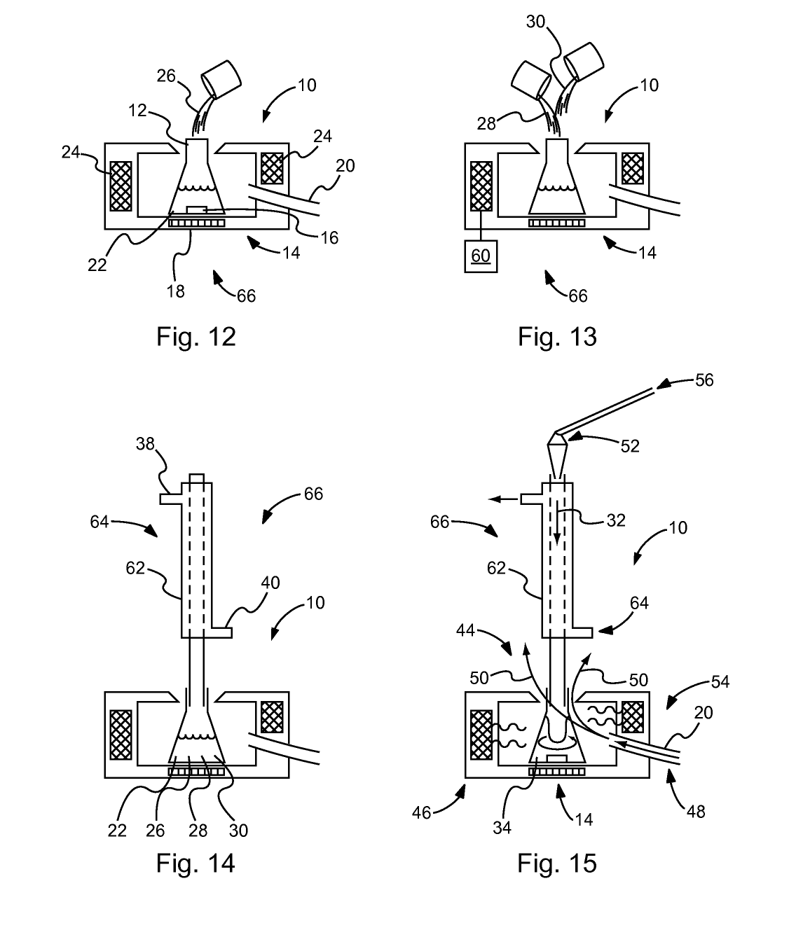

The preferred application of the process of the invention is shown in FIGS. 12 through 16. The process of the invention 10 begins in FIG. 12 by the addition of a pentafluorosulfanyl chloroaromatic compound first reactant 22 to the reaction vessel 12. The reaction vessel 12 is a fluorine resistant vessel. In the example of the preferred embodiment, the reaction vessel 12 is a glass one, compatible for use in the preferred microwave heater, the Discover BenchMate System microwave. FIG. 12 shows a reaction vessel 12 in a microwave heater 24. FIG. 12 illustrates the step of adding a pentafluorosulfanyl chloroaromatic compound first reactant 22 to the reaction vessel 12, and the step of adding a second reagent 26, which is a fluoride reagent. The pentafluorosulfanyl chloroaromatic first reactant 22 is selected from the group comprising compounds produced by chlorination of pentafluorosulfanylbenzene or pentafluorosulfanylnaphthalene; or by direct fluorination of bis(chlorophenyl)disulfides, and bis(chloronapthalene) disulfides.

An activating substituent, like CN (nitrite), NO₂ (nitro), CF₃ (trifluoromethyl) and F (fluoro) substitution on the benzene ring, enhances the efficiency of Halex substitution, and can be converted to other functional groups. While chlorobenezonitriles and chloronitrobenzenes have high boiling points, between 200 and 300° C., the F and CF₃ substituted analogs have lower boiling points between 100-200° C., and are being used for the experiments. Pentafluorosulfanyl-4-chlorobenzene has proven to be an acceptable and preferred first reactant 22. The reaction assembly 66 includes a stirring feature 14, in the form of a magnetic stir bar 16, and a magnetic stir motor 18. Other reaction assemblies could have other forms of stirring, such as an impeller, or other stirring means known in the chemical field.

The reaction assembly 66 includes a cooling system 20, in the form of a flow of cooled air 50 which flows around the outside of the reaction vessel 12. Other cooling systems known in the chemical field would also be suitable for use in the process of the invention.

The second reactant 26 is selected from the group comprising basic fluorides like KF, and acidic fluorides like SbF₃ and AlF₃, for introducing fluorine atoms into organic compounds. Fluorinated organics are huge synthetic ingredients for the production of fine pharmaceuticals and agrochemicals, and have market worth estimated at more US $5 billion.

Although basic KF is widely applied in conventional industrial HALEX processes, acidic AlF₃ and SbF₃ have no dipoles and may be better suited for potentially energy efficient microwave-assisted Halex processes of the future.

The reaction assembly 66 of FIG. 12 is a representation, and the other configurations may vary due to the volumes of the reactants.

FIG. 13 shows the step of adding a phase transfer catalyst 28, and the step of adding a solvent 30. The process of the invention utilizes a phase transfer catalyst (PTC), which for this chemistry is a catalyst which facilitates the migration of a reactant in a heterogeneous system from one phase into another phase where reaction can take place. Phase transfer catalysts for the HALEX processes will include all microwave-safe quaternary ammonium and quaternary phosphonium salts. Examples of phase transfer catalysts include tetra-n-butylammonium chloride, tetra-n-butylammonium bromide, cetyltrimethylammonium bromide, cetyltrimethylammonium chloride, tetraphenylphosphonium bromide, tetraphenylphosphonium chloride, tetramethylammonium chloride, and tetramethylammonium bromide.

A solvent 30 is optional and may be selected from the group comprising dimethylsulfoxide (DMSO), N-methylpyrrolidone (NMP), sulfolane, dimethylformamide, and N,N-dimethylacetamide (DMAC), chlorobenzene, dichlorobenzene, trichlorobenzene, xylene, and toluene. The halogen exchange reaction of the invention can be performed in a dipolar aprotic solvent, in a non-polar solvent in the presence of a phase transfer catalyst, or in the absence of a solvent. The process of the invention is applicable for the production of fluoroorganic compounds, including, but not limited to fluoroaliphatics, fluorinated cyclic-aliphatics, fluoroaromatics, and fluorinated heterocyclic rings.

If the characteristics of a dipolar non-polar solvent are required to achieve a higher yield and easy isolation of the product, such may be utilized with the process of the invention. Examples of dipolar solvents include dimethylsulfoxide (DMSO), N-methylpyrrolidone (NMP), sulfolane, dimethylformamide, and N,N-dimethylacetamide (DMAC). The preferred dipolar solvent is sulfolane because it is inexpensive and is stable and its boiling point is above the reaction temperature. Examples of non-polar solvents include chlorobenzene, dichlorobenzene, trichlorobenzene, xylene, and toluene. Trichlorobenzene is preferred as a non-polar solvent, as its boiling point is above the reaction temperature. If an aprotic solvent is used, sulfone is suitable, as is methylpyrrolidinone, which are high and medium microwave absorbers, respectively.

The phase transfer catalyst 28, for instance (C₁₈H₃₇)₃(Me)N⁺Cl⁻, is miscible with the first reactant 22, (chloroaromatic reagent), and together they form a homogenous liquid. The halide exchange between basic fluorides, like KF, CsF, or RbF and chloroaromatic reagents is expected to rapidly produce the analogous chloride products as stable side products. FIG. 13 includes software controller 60 for controlling the heating cycle of the microwave.

FIG. 14 shows the addition of a reflux cooler 62 to the reactor assembly. The reflux cooler 62 includes an inlet 40 and an outlet 38 for the cooling liquid 64 that flows through the reflux cooler 62, with the purpose of cooling and condensing the evaporated reactants.

FIG. 15 shows the assembled reaction assembly 66, including the inert gas purge 56 through inert gas 32 is injected into the system. FIG. 15 illustrates the step 44 of stirring the contents of the reaction vessel, the step 46 applying microwave energy to the contents of the reaction vessel 12, the step 52 of purging the reaction vessel 12 with inert gas 32 and the step 54 of cooling the reaction vessel simultaneous with microwave heating, by initiating airflow 50.

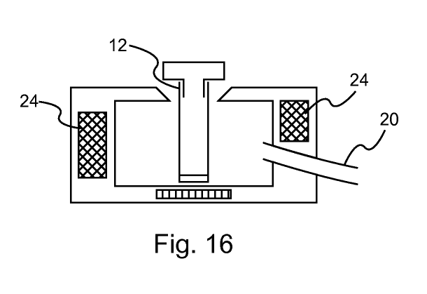

FIG. 16 shows the reaction vessel 12 when performed under a sealed and pressurized setup.

The cooling of the reactants simultaneously with application of highest possible microwave power (50 W-600 W), which forces the system to rapidly attempt to attain the set temperature. The set-temperature is the temperature at which the reaction occurs. The heat doesn't do the magic of reducing the process time as does the unique application of microwave power. Microwave irradiation heats the system, and energizes individual bonds to reaction. The cooling allows continuous application of the beneficial MW power while keeping the solution within the optimum temperature bounds.

After the reaction has proceeded a sufficient time, the reactants are allowed to cool, and the reaction assembly is purged with inert gas 32. After the reactants have cooled, the liquid product 34 is recovered from the reaction vessel 12.

A preferred microwave heater is the Discover BenchMate System microwave fabricated by CEM Instruments of Matthews, N.C. Current commercially available laboratory microwaves can operate up to 400° C. with a glass or quartz reactor. However, fluoride-corrosion resistant reactors are currently operable up to 200° C. (Teflon) and 260° C. (Teflon-PEEK polymer). Many halogen exchange reactions of KF, CsF or RbF were previously performed in glass vessels, taking necessary precautions to prevent moisture in the system. These fluorides will react with trace moisture leading to production of hydrogen fluoride gas that etches glass.

The following are some of the specifications of the Discover BenchMate System microwave which is the preferred microwave heater for the process of the invention. The Discover BenchMate system is a 300-watt laboratory microwave reactor module. The microwave applicator provides a self-tuning feature to insure optimal field tuning to all samples. The platform includes a fluoropolymer sleeved cavity with cavity access port, vacuum fluorescent display with alpha/numeric capabilities, an alpha/numeric keypad, (1) computer port, (1) Ethernet network port, and a detachable power cord. The system is capable of continuous power delivery which can be varied in 1-watt increments. It has dimensions of 14.4 W×17.2 D×8.7H with a weight of 30 lbs. The LabMate Intellivent package comes configured with the following options:

Magnetic Stirring Option

Standard Cooling Option

Infrared Temperature Feedback Control

Intellivent SafeSeal Option

Accessory Kit

This system accommodates vessels ranging in size from a 5-mL tube to a 125 mL round bottom flask −24/40 ground glass joint.

One preferred embodiment of the process of the invention is one that utilizes a software controller for controlling the microwave output over time. The Synergy Software Option is a PC-based software package that allows the user to program, monitor, and control all System functions. The software package communicates via either serial port or Ethernet connection to the various instruments. The package creates, and allows for the full management of, a database for all system reaction data. System methods can be downloaded to the Instrument Modules or uploaded from the Instrument Modules' onboard memory to the software platform. The package is Windows 2000, NT, and Vista compliant. Minimum system requirements are a Pentium P4 class processor running at 1.6 GHz, with 64 Mb of RAM and with at least 120 Mb of available disk space. An Ethernet and serial Cable is included in the option.

The reaction of the process of the invention is preferably carried out between 140-260° C. The maximum operable temperature of a Teflon reactor may restrict the maximum temperature to 200° C. The reactions of KF, CsF and RbF can occur at 200° C.

The reactor system can be conducted at pressures up to 300 PSIG, however, use of a Teflon reactor may reduce the maximum pressure to 120 PSIG.

The process of the invention is exemplified by the following example procedures.

### Example 1

Place 20.2 g (0.116 mol) 3,4-dinitrochlorobenzene, 0.7 g (0.002 mol) (C₈H₁₇)₃N⁺Cl⁻, a phase transfer catalyst (about 6% mol chloroaromatic reagent). Freeze the chloroaromatic reagent, and pull vacuum to remove air and moisture from the system. Purge the system with nitrogen to atmospheric pressure. While maintaining inert atmosphere in the 125 mL reactor, add 6.8 g (0.117 mol) potassium fluoride—a solid fluorinating agent, a magnetic stir bar, and fit the reactor to a 24″ reflux condenser that is fitted to a source of constant purging by an inert gas. Program the software to set reaction profile and power cut off limit. Microwave power was set at 300 W, and this was accompanied by flow of dry air regulated to 20 PSIG from a compressed cylinder at 1 standard liter per minute (SLPM), power will be required. Ensure vigorous stirring of the reaction. After adequate reaction time has lapsed, cool down the system to room temperature. Purge the system with N₂ or Helium. Aliquot samples were removed at 1 h, 2 h, 5 h, and 9 h process time and analyze raw product samples by GC to reveal yields of 24, 35, 44, and 92 percent yields, respectively. This result compared to 13.4 percent yield of the expected product-3-chloro-4-fluoronitrobenzene—by conventionally heating the reaction for 9 h, in U.S. Pat. No. 5,545,768 (1996).

### Example 2

In this work, pentafluorosulfanyl 4-chlorobenzene and pentafluorosulfanyl 4-fluorobenzene were prepared in 39-42% by direct fluorination of the respective bis(4-halophenyl) disulfide by using 10% Fluorine in nitrogen mixture at low temperatures. The products in the figure below were characterized by their boiling points.

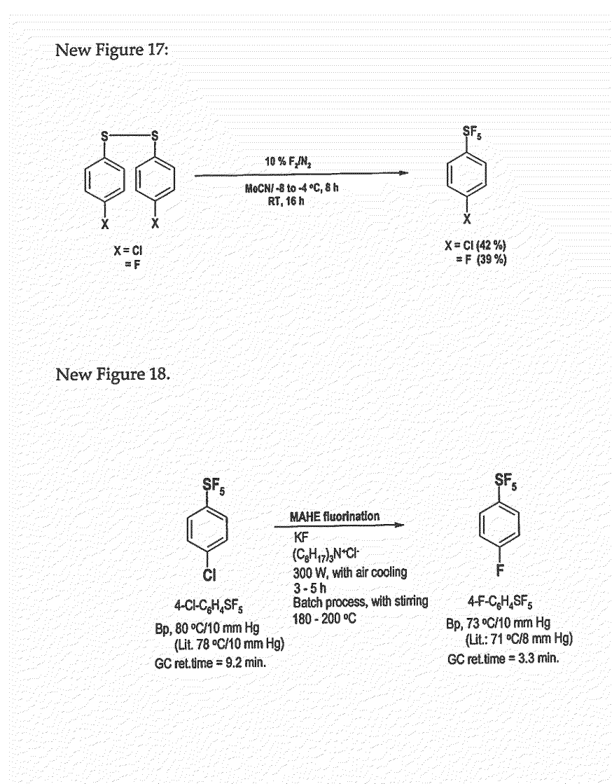

FIG. 17 shows the production of pentafluorosulfanyl 4-halobenzene by direct fluorination of bis(4-halophenyl)disulfide.

Their individual retention times were obtained by elution of volatile solution on a gas chromatograph, and used to calibrate the method for determination of yields of microwave-assisted halogen exchange (MAHE) fluorination process in the FIG. 18.

FIG. 18 shows the MAHE fluorination of pentafluorosulfanyl 4-bromobenzene.

0.2 g (0.000839 mol) 4-Chlorophenyl sulfurpentafluoride, (4-C₁—C₆H₄SF₅); 0.4 g (0.0069 mol) spray dried potassium fluoride; 0.05 g, (0.000129 mol) (C₈H₁₇)₃N⁺C₋; 3 mL N-methylpyrrolidinone (NMP); and a 1-cm long magnetic stirrer were placed in a 10 mL reactor in an inert atmosphere. The CEM Intellivent™ cap, a safety vent at 300 PSIG, was carefully placed as reactor cover. The mixture consisted of clear liquid solution of A in xylene or nitrobenzene over solid white potassium fluoride and magnetic stirrer. The glass reactor was then placed into the single mode microwave chamber of CEM Discover™ instrument The CEM Synergy™ software was used to control and program power, stirring, temperature, and cooling inputs of the process method. The method for this process included the following settings: power=300 W, stirring=ON, temperature=180° C.; chamber cooling=ON. The method controlled the start, stop, and program the duration of the application of microwave power. At the end of the 3 h process, and subsequent cooling, the intellivent cap was carefully opened to reveal a brown mixture. The solution was extracted with 50 mL diethyl ether. The organic content was washed twice with 20 mL water in a separating funnel, and dried by using magnesium sulfate. The crude mixture was analyzed by Gas chromatography (GC). Further, the diethyl ether solution mixture of the byproducts was evaporated under vacuum, dissolved in deuterated chloroform (CDCl₃), and analyzed by ¹⁹F nuclear magnetic resonance (NMR). The spectrum shows that the sulfurpentafluoride (SF₅) group, as signals at 62.6 ppm (doublet) and 82.8 ppm (quintet), was not destroyed by the MAHE fluorination process.

Preliminary recording of GC retention time for 4-chlorophenyl sulfurpentafluoride (FIG. 1), starting material, and 4-fluorophenyl sulfurpentafluoride (FIG. 2), the final product of MAHE fluorination, were reference calibration used for the determination of the process yields of 00%. 4-Chlorophenyl sulfurpentafluoride (bp, 78° C./10 mm Hg) and 4-fluorophenyl sulfurpentafluoride (bp, 72° C./80 mm Hg) were prepared from direct fluorination of the respective bis(4-halophenyl)disulfide, according to the method in Tetrahedron 2000, and characterized by their boiling point and ¹⁹F NMR spectrum.

### Example 3

5 g (0.021 mol) 4-Chlorophenyl sulfurpentafluoride, (4-Cl—C₆H₄SF₅); 10 g (0.172 mol) spray dried potassium fluoride; 0.34 g, (0.00129 mol) 18-Crown-6; 40 mL N-methylpyrrolidinone (NMP, an average absorber of microwave energy); and a 3-cm long magnetic stirrer were placed in a 125 mL reactor in an inert atmosphere. The flask was carefully fitted with a reflux condenser and constant nitrogen purge at 50 ml/min at 1 atm. At the beginning, the mixture consisted of clear liquid solution in sulfolane over solid white potassium fluoride and magnetic stirrer. The glass reactor was then placed into the single mode microwave chamber of CEM Discover™ instrument The CEM Synergy™ software was used to control and program power, stirring, temperature, and cooling inputs of the process method. The method for this process included the following settings: power=300 W, stirring=ON, temperature=180° C.; chamber cooling=ON. The method controlled the start, stop, and program the duration of the application of microwave power. At the end of the 5 h, and subsequent cooling, the condenser was carefully removed, and the flask taken out of the microwave chamber to reveal a brown mixture. The solution was extracted with 150 mL diethyl ether. The organic content was washed twice with 60 mL water in a separating funnel, and dried by using magnesium sulfate. The crude mixture was analyzed by Gas chromatography (GC), and the results are shown in FIG. 6. The determination of the process yields of 78% in FIG. 6 was performed in a manner similar to the description in Example 2.

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. An improved process for production of fluoroaromatic compounds by halogen exchange, comprising the steps of:
providing a reaction vessel with a stirring feature for stirring reactants, a cooling system, and a microwave heating system;
adding a fluoride reagent as a fluorinating agent to said reaction vessel as a reactant;
adding a phase transfer catalyst;
stirring the contents of the reactor vessel;
applying microwave energy to heat said reactants to a predetermined temperature and to energize individual bonds to reaction;
cooling the reaction vessel simultaneously with said microwave heating system, to allow increased microwave energy to be applied to the reactants while keeping the reactants below a selected temperature;
allowing the fluorination reaction to continue for a predetermined amount of time;
cooling the reactants and reaction vessel;
purging the reaction vessel with an inert gas; and
recovering the liquid product from the reaction vessel.

2. An improved process for production of pentafluorosulfanyl fluoroaromatic compounds by halogen exchange, comprising the steps of:
providing a reaction vessel with a stirring feature for stirring reactants, a cooling system, and a microwave heating system;
adding a pentafluorosulfanyl chloroaromatic compound first reactant to the reaction vessel;
adding a fluoride reagent as a fluorinating agent to said reaction vessel as a second reactant;
adding a phase transfer catalyst to the mixture of first and second reactants;
activating stirring to the reactor vessel;
applying microwave energy to heat said reactants and to energize individual bonds to reaction;
activating said cooling system simultaneously with said microwave heating system, to allow increased microwave energy to be applied to the reactants while keeping the reactants below a selected temperature;
allowing the fluorination reaction to continue for a predetermined amount of time;
allowing the reactants and reaction vessel to cool;
purging the reaction vessel with an inert gas; and
recovering the liquid product from the reaction vessel.

3. The improved process for production of fluorinated organic compounds by halogen exchange of claim 2, in which said cooling step continues until the product reaches room temperature.

4. The improved process for production of fluorinated organic compounds by halogen exchange of claim 2, which includes the step of adding a solvent to said reaction vessel before heating said reaction vessel.

5. The improved process for production of fluorinated organic compounds by halogen exchange of claim 2, in which said pentafluorosulfanyl chloroaromatic compound is selected from the group comprising pentafluorosulfanyl chloroaromatic, chloro(organic substituted)aromatic, chloro(halo substituted)aromatic, polychloroaromatic, polychloro(substituted)aromatic, and chloro(polysubstituted)aromatic compounds.

6. The improved process for production of fluorinated organic compounds by halogen exchange of claim 2, in which said fluoride reagent is selected from the group comprising KF, CsF and RbF.

7. The improved process for production of fluorinated organic compounds by halogen exchange of claim 2, in which said solvent is an aprotic solvent.

8. The improved process for production of fluorinated organic compounds by halogen exchange of claim 2, which includes the step of activating a software controller for the control of said microwave heating system.

9. The improved process for production of fluorinated organic compounds by halogen exchange of claim 2, in which the step of activating a stirring to the reaction vessel further comprises adding a magnetic stir bar to said reaction vessel.

10. The improved process for production of fluorinated organic compounds by halogen exchange of claim 2, in which the step of cooling is by use of an enhanced airflow through the reaction vessel;

11. The improved process for production of fluorinated organic compounds by halogen exchange of claim 2 in which the reactants are maintained at a temperature of approximately 140-260 C during the reaction.

12. The improved process for production of fluorinated organic compounds by halogen exchange of claim 2, which further comprises the step of freezing the chloroaromatic reagent before heating begins in the reaction vessel.

13. The improved process for production of fluorinated organic compounds by halogen exchange of claim 2 which further comprises the step of pressurizing the reaction vessel to up to 300 psia during the reaction.

14. The improved process for production of fluorinated organic compounds by halogen exchange of claim 2 which further comprises flushing the reaction vessel with an inert gas such as nitrogen, helium, or argon.

15. The improved process for production of fluorinated organic compounds by halogen exchange of claim 2 in which said step of recovering the liquid product comprises filtering the liquid from the solid phases, followed by distillation.

16. The improved process for production of fluorinated organic compounds by halogen exchange of claim 2 in which said step of recovering the liquid product comprises extracting an ionic salt product into an aqueous phase.

17. The improved process for production of fluorinated organic compounds by halogen exchange of claim 2 in which said phase transfer catalyst is selected from the group of thermally stable neutral or ionic compounds, comprising 18-Crown-6, traditional ionic liquids, tetraalkylammonium salts, teteraalkylphosphonium salts, tetraarylphosphonium salts, onium salts, delocalized cations, 2-azaallenium salts, carbophosphazenium salts, aminophosphonium salts, and diphosphazeium salts, etc.

18. The improved process for production of fluorinated organic compounds by halogen exchange of claim 2 in which said process is catalyzed by at least one phase transfer catalyst.

19. The improved process for production of fluorinated organic compounds by halogen exchange of claim 2 in which said pentafluorosulfanyl (SF₅) group on the products of the halogen exchange process is stable to microwave energy irradiation in a reagent mixture consisting of <15% ionic catalyst content
